(19) United States
(12) Patent Application Publication
(10) Pub. No.: US 2016/0109459 A1
(43) Pub. Date: Apr. 21, 2016

(54) Compositions And Methods For Heparan Sulfate As A Biomarker For Transplant Rejection

(71) Applicant: Duke University, Durham, NC (US)

(72) Inventors: Todd V. Brennan, Durham, NC (US); Yiping Yang, Chapel Hill, NC (US)

(21) Appl. No.: 14/398,314

(22) PCT Filed: May 1, 2013
(86) PCT No.: PCT/US2013/039086

§ 371 Date: Oct. 31, 2014

Related U.S. Application Data
(60) Provisional application No. 61/641,043, filed on May 1, 2012.
(60) Provisional application No. 61/660,914, filed on Jun. 18, 2012.

(51) Int. Cl. G01N 33/68; A61K 38/57; A61K 45/06; A61K 38/51

(52) U.S. Cl. CPC G01N 33/6893; A61K 38/51; A61K 38/57; A61K 45/06; G01N 2800/245; C12Y 402/02007

(57) ABSTRACT

The present disclosure provides methods of identifying transplant rejection through the use of heparan sulfate as a biomarker. Method also comprise treating and/or preventing transplant rejection in a subject comprising administering to the subject a heparan sulfate inhibitor, thereby treating and/or preventing the development of immune-mediated injury following transplantation.

### CROSS REFERENCE TO RELATED APPLICATIONS

This application claims the benefit of priority to U.S. Provisional Patent Application Ser. Nos. 61/641,043 filed May 1, 2012 and 61/660,914 filed Jun. 18, 2012, which are incorporated herein by reference in its entirety.

### FEDERAL FUNDING LEGEND

This invention was produced in part using funds from the Federal Government under NIH Grant Nos: CA136934 and CA047741. Accordingly, the Federal Government has certain rights to this invention.

### BACKGROUND

Allogeneic hematopoietic stem cell transplantation (Allo-HSCT) is a potentially curative therapy for many types of hematologic malignancies and nonmalignant hematologic diseases. (Copelan, E. A. (2006) N. Engl. J. Med., 354:1813-26). However, graft-versus-host disease (GVHD) remains a prevalent and serious side effect that limits the effectiveness of this therapy. (Ferrara, J. L. et al. (2009) Lancet. 373:1550-61; Welniak, L. A., Blazar, B. R., & Murphy, W. J. (2007) Annu. Rev. Immunol. 25:139-170). Although T-cell depletion (TCD) of the bone marrow graft results in decreased rates of GVHD (Devine, S. M. et al. Biol. Blood Marrow Transplant (2011); Hale, G. & Waldmann, H. (1994) Bone Marrow Transplant 13:597-611), it is associated with general immunodeficiency that predisposes recipients to higher rates of viral and fungal infections (van Burik, J. A. et al. (2007) Biol. Blood Marrow Transplant 13:1487-98), as well as increased tumor recurrence rates (Zhang, P., Chen, B. J. & Chao, N. J. (2011) Immunol. Res. 49:49-55). In fact, some level of GVHD may be beneficial to the recipient by being associated with a more robust graft-versus-tumor (GVT) response as demonstrated by lower tumor recurrence rates in these patients. (Goldstein, S. C. & Porter, D. L. (2010) Expert Rev. Hematol. 3:301-14).

Innate immunity is the rapid response system by which a host can recognize and respond to infection or tissue injury. The rapidity of the innate response is due to fixed pattern recognition receptors (PRRs) that are naturally abundant and poised for immediate response. Toll-like receptors (TLRs), the best characterized family of PRRs, were originally characterized for their ability to respond to exogenous “pathogen-associated molecular patterns”, or PAMPs, that include bacterial lipopolysaccharide (LPS), bacterial diacylated and triacylated lipopeptides, bacterial flagellin, bacterial and viral unmethylated CpG-containing DNA motifs, and viral single- and double-stranded RNA. (Akira S., Uematsu, S., & Takeuchi, O. (2006) Cell 124:783-801. In addition to PAMPs, TLRs also recognize endogenous “damage-associated molecular patterns”, or DAMPs. Examples include proteins such as heat-shock protein 60 (Hsp60), Hsp70, surfactant protein A, high mobility group box 1 (HMGB1), fibrinogen and fibronectin, as well as polysaccharides such as hyaluronan and heparan sulfate. (Beg, A. A. (2002) Trends Immunol. 23:509-12; Tsan, M. F. & Gao, B. (2004) J. Leukoc. Biol. 76:514-19).

It has become increasingly apparent that TLRs play a critical role in shaping effective adaptive immune responses in a variety of conditions such as infection, cancer, and autoimmunity. (Kawai, T. & Akira, S. (2010) Nat. Immunol. 11:373-84; Huang, X, & Yang, Y. (2010) Expert Opin. Ther. Targets 14:787-96). It has also been shown that TLR4 and MyD88 deficiencies are protective against acute rejection in the setting of solid organ transplantation. (Chen, L. et al. (2006) Am. J. Transplant 6:2282-91; Goldstein, D. R. et al. (2003) J. Clin. Invest. 111:1571-78; Palmer, S. M. et al. (2003) Am. J. Respir. Crit. Care Med. 168:628-32). Similarly, stimulation of TLR9 with CpG oligodeoxynucleotides (ODN) markedly accelerates GVHD lethality (Taylor, P. A. (2008) Blood 112:3508-16), suggesting a role for TLR pathways in modulating GVHD. Since the onset of GVHD usually occurs in the absence of obvious exogenous stimuli such as bacterial or viral infections, the role of endogenous TLR agonists in the development of GVHD was investigated.

Heparan sulfate (HS), a ubiquitous component of the extracellular matrix, was determined to be a potent stimulator of T cell alloreactivity in vitro. The stimulatory effect of HS was dependent on an intact TLR4 pathway in dendritic cells (DCs), but not in alloreactive T cells, by promoting DC maturation and function. When the in vivo relevance of the observed effect of HS on the alloreactive T cell response in a murine model of Allo-HSCT were tested, serum levels of HS were highly elevated at the onset of clinical symptom of GVHD. Suppression of HS release by the serum protease inhibitor, al-antitrypsin (A I AT), decreased the levels of serum HS, inhibited the activation of donor-derived T cells in vivo, and resulted in a significant improvement in GVHD and survival. Conversely, increasing serum levels of HS during GVHD using a HS mimetic increased donor T cell proliferation in vivo and GVHD severity. In human recipients of Allo-HSCT, increased serum HS levels were directly correlated to the severity of GVHD.

Likewise, HS was investigated for its role as an endogenous stimulator of alloimmunity and as an early marker of immune injury in a mouse heart transplant (tx) model. Lymphocytic tissue infiltration is the hallmark of immune-mediated injury of organ transplants. Vascular diapedesis and intercellular migration of lymphocytes require the breakdown of extracellular matrix.

The studies herein demonstrate that HS can promote the alloreactive T cell response and increase the severity of GVHD, and suggest that strategies to block HS release may have therapeutic potential in the prevention of GVHD. Additionally, the results herein demonstrate a role of HS as a marker of tissue injury in the setting of organ transplantation and in promoting alloimmunity by serving as an endogenous activator of innate immune pathways. Elevations in serum or urine HS may serve as an early biomarker of acute cellular rejection. Blocking extracellular matrix breakdown may inhibit lymphocytic tissue infiltration and reduce T cell activation.

### SUMMARY OF THE INVENTION

The present disclosure is based, in part, on the surprising discovery that heparan sulfate (HS) can activate Toll-like receptor 4 on dendritic cells (DC) in vitro, leading to the enhancement of DC maturation and alloreactive T cell responses Inhibiting HS with a serine protease inhibitor leads to a reduction in alloreactive T cell responses following transplantation, and a reduction in graft-versus-host disease severity.

One aspect of the present disclosure provides a method of treating or ameliorating an innate immune injury following organ, tissue, or cellular transplant in a subject comprising, consisting of, or consisting essentially of administering to the subject a heparan sulfate inhibitor as described herein, thereby treating the innate immune injury. In another aspect, the disclosure provides methods of treating or ameliorating an injurious condition associated with elevated heparan sulfate comprising, consisting of, or consisting essentially administering an inhibitor that decreases serum heparan sulfate to a therapeutically effective level, to a subject that was the recipient of a transplanted organ, tissue, or cells.

Another aspect of the present disclosure provides a method of preventing an innate immune injury following organ, tissue, or cellular transplant from developing in a subject comprising, consisting of, or consisting essentially of administering to the subject a heparan sulfate inhibitor as described herein, thereby preventing the innate immune injury from developing. In another aspect, the present disclosure provides methods of preventing an injurious condition associated with elevated heparan sulfate comprising, consisting of, or consisting essentially of administering an inhibitor that, decreases serum heparan sulfate to a therapeutically effective level, to a subject that was the recipient of a transplanted organ, tissue, or cells.

Yet another aspect of the present disclosure provides methods of treating or preventing graft-versus-host-disease (GVHD) in a subject comprising, consisting of, or consisting essentially of administering to the subject a heparan sulfate inhibitor as described herein, thereby treating the GVHD.

Yet another aspect of the present disclosure provides methods of treating GVHD and/or preventing GVHD from developing in a subject comprising, consisting of, or consisting essentially of administering to the subject a serine protease inhibitor as described herein, the inhibitor being capable of reducing serum levels of heparan sulfate. In some embodiments, the serine protease inhibitor is al-antitrypsin.

In some embodiments, the innate immune injury is characterized by increased serum concentrations of heparan sulfate. In yet other embodiments, the innate immune injury is selected from the group consisting of inflammation, graft rejection, GVHD, and acute cardiac allograft rejection. In certain embodiments, the innate immune injury comprises GVHD.

The disclosure also provides compositions comprising an inhibitor that decreases serum heparan sulfate to a therapeutically effective level in a transplant recipient subject, and a pharmaceutically acceptable carrier.

Another aspect of the present disclosure is a method of diagnosing an injurious condition that is associated with elevated heparan sulfate in a subject that was the recipient of a transplanted organ, tissue, or cells, by collecting a biological sample from the subject and determining the serum concentration of heparan sulfate, where the concentration of heparan sulfate directly correlates with the severity of the heparan sulfate-mediated immune injury.

Another aspect of the present disclosure provides for all that is disclosed and illustrated herein.

### BRIEF DESCRIPTION OF THE DRAWINGS

The foregoing aspects and other features of the invention are explained in the following description, taken in connection with the accompanying drawings, wherein:

FIG. 1 is a schematic demonstrating that several extracellular matrix components have been shown to activate toll-like receptors.

FIG. 2 demonstrates that HS is a potent stimulator of alloreactive T cell responses though the TLR4 and MyD88-dependent activation of DCs. FIG. 2A is a graph demonstrating that TLR and NLR agonists were assayed in allogeneic T cell proliferation assay between purified T cells (2×10⁵/well) from C57BL/6 mice and bone marrow-derived BALB/c DCs (2.5×10⁴/well). Cells were co-cultured either alone (media) or in the presence of LPS (100 ng/mL), Pam3CSK4 (2 μg/mL), hyaluronan (HA) (100 μg/mL), sonicated-HA (sHA) (100 μg/mL), fibronectin (FN) (100 μg/mL), fibrinogen (Fbn) (100 μg/mL), heparan sulfate (HS) (100 μg/mL), HSP70 (5 μg/mL), HMGB1 (1 μg/mL), C12-iE-DAP (1 μg/mL), or L18-MDP (1 μg/mL) for 72 hours, and then pulsed ³H-thymidine for 16 hours. Proliferation was determined by ³H incorporation and results are expressed as cpm±SEM. Baseline alloreactivity is indicated by the dotted line; *p<0.05 compared with media alone. FIG. 2B is a graph demonstrating that proliferation performed as in (A) +/− the addition of the LPS inhibitor, polymyxin B (PMB; 10 ug/mL) *p<0.05. FIG. 2C is a graph demonstrating that the proliferation assay performed as in (A) with purified responder T cells (R) from either WT (+) or MyD88^{-/-} (−) C57BL/6 mice were co-cultured with DC stimulators (S) from either WT (+) or MyD88^{-/-} (−) BALB/c mice; *p<0.05 compared with media alone in S+/R+ group. FIGS. 2D & 2E are graphs demonstrating proliferation and IFN-γ production in proliferation assays performed as in (A) using WT, TLR4^{-/-}, and MyD88^{-/-} BALB/c DCs as stimulators and purified C57BL/6 T cells as responders (*p<0.05). Results are representative of three independent experiments.

FIG. 3 demonstrates that HS promotes DC maturation and production of pro-inflammatory cytokines via the TLR4-MyD88 pathway. FIG. 3A is a graph showing WT, TLR4^{-/-}, or MyD88^{-/-} BALB/c DCs (2×10⁵/well) were stimulated with LPS (100 ng/mL), HS (100 μg/mL), or Pam3CSK4 (2 μg/mL), or left unstimulated (media) for 24 hours, and measured for surface expression of co-stimulatory molecules CD40 and CD80 by FACS analysis. FIGS. 3B and 3C are graphs showing WT, MyD88^{-/-}, and TLR4^{-/-} BALB/c cultured DCs were co-cultured with media alone, LPS, HS, or Pam3CSK4 as in (A) and culture supernatants were tested for IL-6 (B) and IL-12 (C) by ELISA. Data are representative of three independent experiments; *p<0.05 compared with media alone. FIGS. 3D and 3E are graphs showing and assay of DC production of IL-6 and IL-12+/− the addition of the LPS inhibitor, PMB (10 μg/mL) following stimulation with media, HS, or LPS; *p<0.05.

FIG. 4 is a graph showing that the intracellular adaptor molecule, TRIF, has a minor role in HS induction of IL-6 expression by DCs. ELISA analysis of IL-6 production by WT, TLR4^{-/-}, TRIF^{-/-}, and MyD88^{-/-} cultured C57BL/6 DCs (2×105/well) incubated with HS (25 μg/mL), LPS (100 ng/mL) or CpG (10 μg/mL) for 24 hrs, *p<0.05.

FIG. 5 demonstrates that TLR4 is sufficient for HS induced activation of NF-κ B and IL-8 expression. HEK cell lines stably expressing CD14 and MD2 alone (HEK MD2-CD14), or co-expressed with human TLR2 (HEK TLR2-MD2-CD14) or human TLR4 (HEK TLR4-MD2-CD14) were transfected with a plasmid encoding firefly luciferase (F) under control of the NF-κB promoter along with a plasmid expressing Renilla luciferase (R) under control of the thymidine kinase promoter as a transfection control. Cell lines were cultured for 6 hours with media alone, LPS (100 ng/mL), HS (100 μg/mL), or Pam3CSK4 (2 μg/mL). FIG. 5A is a graph showing the ratio of F/R was measured by dual luciferase reporter assay to determine NF-κB activation of lysed cells, *p<0.05 compared with media alone. FIG. 5B is a graph showing results supernatants analyzed for IL-8 production by ELISA. FIG. 5C is a graph showing IL-8 production in response to LPS and HS+/−PMB in HEK TLR4-MD2-CD14 cells, *p>0.05. FIG. 5D is a graph showing +/− heparanase (Hpase) in HEK TLR4-MD2-CD14 cells, *p<0.05. Experiments were performed in triplicate or quadruplicate. Results are representative of two to three independent experiments.

FIG. 6 demonstrates that serum HS is highly elevated at the onset of GVHD. Lethally irradiated BALB/c recipients received either 1×10⁷ B10.D2 TCD-BM only (Allo-BM), 1×10⁷ B10.D2 TCD-BM and 5×10⁶ B10.D2 LCs (Allo-BM+LC), or 1×10⁷ BALB/c TCD-BM and 5×10⁶ BALB/c LCs (Syn-BM+LC). FIG. 6A is a graph showing HS concentrations following transplantation as determined by ELISA at the indicated time points; n=2-5 samples per time point; *p<0.05 comparing Allo-BM+LC and Allo-BM at the indicated time point. FIG. 6B is a graph showing the half maximal effective concentration (EC₅₀) of HS on DC stimulation, BALB/c DCs (2×10⁵/well) following 24 hr of culture with differing concentrations of HS in triplicate and IL-6 production was tested by ELISA. Results are representative of three independent experiments.

FIG. 7 demonstrates that A1AT decreases serum HS levels and improves the outcome of GVHD following Allo-HSCT. FIG. 7A is a graph showing serum HS concentrations at the indicated time points following Allo-HSCT (B10.D2→BALB/c; 1×10⁷ B10.D2 TCD-BM and 5×10⁶ B10.D2 LC) treated with A1AT (2 mg) or PBS every 3 days by i.p. injection, starting 1 day prior to transplantation; n=3 per data point, as determined by ELISA assay; *p<0.05. FIG. 7B is a graph showing survival and FIG. 7C is a graph showing GVHD clinical score of Allo-BM only (n=5) or Allo-BM+LC treated with A1AT (n=8) or PBS (n=5). Data is from one of two independent experiments with identical results. FIG. 7D is a graph demonstrating GVHD pathology score and FIG. 7E is a representative H&E histology of BALB/c recipients of B10.D2 (Allo) TCD-BM+LC treated with PBS or with A1AT (n=6 per group; bar equals 100 μM; *p=0.05). FIG. 7F is a graph demonstrating survival of lethally irradiated C57BL/6 recipients of 1×10⁷ C3H.SW TCD-BM (Allo-BM) and 5×10⁶ C3H.SW LC administered A1AT (n=10) or PBS (n=9). FIG. 7G is a graph demonstrating improvement in GVHD survival by Al AT is dependent on host TLR4 expression. Survival of BALB/c recipients of Allo-BM+LC from B10.D2 donors (n=11) and TLR4^{-/-} BALB/c recipients of Allo-BM+LC from B10.D2 donors administered A1AT (n=8) or PBS (n=14) every 3 days by intraperitoneal injection, starting 1 day prior to transplantation.

FIG. 8 demonstrates that A1AT treatment decreases alloreactive T cell responses in Allo-HSCT recipients. BALB/c Thy1.2 recipient of B10.D2 Thy1.1 Allo-BM+LC were treated with i.p. injections of A1AT (2 mg in PBS) or PBS control every 3 days, beginning 1 day prior to HSCT. FIG. 8A are graphs demonstrating splenocytes that were FACS analyzed for BrdU incorporation and IFN-γ production six days after transplant and pulsed with BrdU. Positive FACS gates were set by isotype antibody staining and plots are representative of 5 mice. FIG. 8B is a graph showing averages and SEM of Thy1.1.⁺T cells positive for BrdU, *p<0.05. FIG. 8C is a graph showing averages and SEM of Thy1.1.⁻T cells positive for IFN-γ, *p<0.05.

FIG. 9 demonstrates that the HS mimetic increases serum HS levels and increases CD8 T cell proliferation in Allo-HSCT recipients. BALB/c recipient of B10.D2 Allo-BM+LC were treated with subcutaneous injections of the HS mimetic, PG545 (20 mg/kg in PBS), or PBS control once weekly, beginning 1 day prior to Allo-HSCT. FIG. 9A is a graph demonstrating serum HS levels on the indicated days following transplant as determined by ELISA assay; n=2-4 samples each, *p<0.05. FIG. 9B is a graph demonstrating BrdU uptake by CD8 T cells 6 days after Allo-HSCT. Average and SEMare plotted; n=3 per group; *p<0.05. FIG. 9C is a graph showing survival analysis of Allo-BM+LC+PG545 (n=10) compared to Allo-BM+LC+PBS (n=10).

FIG. 10 demonstrates the persistence of recipient MHC class II expressing cells following allogeneic HSCT. Lethally irradiated C57BL/6 recipients received 10⁷ TCD-BM+10⁶ LC from either allogeneic BALB/c donors or syngeneic C57BL/6 donors. Fourteen days after transplant, recipient mice were injected with 2×10⁶ CFSE-labeled lymphocytes from 4C TCR-tg mice (direct allospecificity towards the BALB/c MHC class II molecule, I-A^{d}) that were on the Ly5.1 congenic background. Recipient intrahepatic lymphocytes were harvested 3 days later and FACS analyzed. FIG. 10A is a schematic of the experiment. FIG. 10B is a graph showing FACS gates for detection of 4C TCR-tg T cells and CFSE analysis. Results shown are representative of 4 mice in each group.

FIG. 11 demonstrates that HS is elevated in serum samples of human Allo-HSCT recipients with GVHD. FIG. 11A is a graph demonstrating serum samples from Allo-HSCT recipients were tested for HS by ELISA. Patients were divided into 3 groups: no GVHD (Grade 0, n=8), mild GVHD (Grade I-II, n=17), and moderate to severe GVHD (Grade III-IV, n=11), *p=0.003, **p=0.0009, ***p=0.01. FIG. 11B is a graph showing serum HS levels relative to time of diagnosis of GVHD in patients with Grade I-II and Grade III-IV GVHD. Average+/−SEM plotted, *p=0.01.

FIG. 12 demonstrates acute cardiac rejection following heart transplant. FIG. 12A is a graph showing that serum HS is increased at onset of acute cardiac rejection in mice. FIG. 12B is a graph showing that serum HS is increased at onset of acute cardiac rejection in humans. FIG. 12C is a histology image showing that HS is degraded at sites of lymphocyte (LC) infiltration.

FIG. 13 is a graph showing WT, MyD88^{-/-}, and TLR4^{-/-} BALB/c cultured DCs that were co-cultured with media alone, LPS, HS, or Pam3CSK4 and tested for TNF-α as determined by ELISA assay.

### DETAILED DESCRIPTION OF THE INVENTION

For the purposes of promoting an understanding of the principles of the present disclosure, reference will now be made to preferred embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

Articles “a” and “an” are used herein to refer to one or to more than one (i.e. at least one) of the grammatical object of the article. By way of example, “an element” means at least one element and can include more than one element.

Unless otherwise defined, all technical terms used herein have the same meaning as commonly understood by one of ordinary skill in the art to which this disclosure belongs.

As used herein, the term “subject” is intended to include human and non-human animals. Exemplary human subjects include a human patient having a disorder, e.g., a disorder described herein, or a normal subject. The term “non-human animals” includes all vertebrates, e.g., non-mammals (such as chickens, amphibians, reptiles) and mammals, such as non-human primates, domesticated and/or agriculturally useful animals (such as sheep, dogs, cats, cows, pigs, etc.), and rodents (such as mice, rats, hamsters, guinea pigs, etc.).

One aspect of the present disclosure provides a composition comprising an inhibitor that decreases serum heparan sulfate to a therapeutically effective level in a transplant recipient subject, and a pharmaceutically acceptable carrier.

“Pharmaceutically acceptable,” as used herein, pertains to compounds, materials, compositions, and/or dosage forms which are, within the scope of sound medical judgment, suitable for use in contact with the tissues of a subject (e.g. human) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation.

Another aspect of the present disclosure provides a method of treating or ameliorating an innate immune injury following organ, tissue, or cellular transplant in a subject comprising, consisting of, or consisting essentially of administering to the subject a heparan sulfate inhibitor, thereby treating the innate immune injury. Methods claimed herein include the use of the composition comprising an inhibitor that decreases serum heparan sulfate to a therapeutically effective level in a transplant recipient subject, and a pharmaceutically acceptable carrier.

Yet another aspect of the present disclosure provides a method of preventing an innate immune injury following organ, tissue, or cellular transplant in a subject comprising, consisting of, or consisting essentially of administering to the subject a heparan sulfate inhibitor, thereby treating the innate immune injury. Methods claimed herein include the use of the composition comprising an inhibitor that decreases serum heparan sulfate to a therapeutically effective level in a transplant recipient subject, and a pharmaceutically acceptable carrier.

“Effective amount,” as used herein, refers to a dosage of the compounds or compositions effective for eliciting a desired effect. This term as used herein may also refer to an amount effective at bringing about a desired in vivo effect in an animal, mammal, or human, such as reducing proliferation of a cancer cell. In certain embodiments, the effective amount is measured by the concentration of serum heparan sulfate. In one embodiment, the concentration of serum heparan sulfate directly correlates to the severity of the innate immune injury, where the innate immune injury is GVHD.

As used herein, the term “treat” or “treating” a subject having a disorder refers to administering a regimen to the subject, e.g., the administration of a heparan sulfate inhibitor-based therapeutic, such that at least one symptom of the disorder is cured, healed, alleviated, relieved, altered, remedied, ameliorated, or improved. Treating includes administering an amount effective to alleviate, relieve, alter, remedy, ameliorate, improve or affect the disorder or the symptoms of the disorder. The treatment may inhibit deterioration or worsening of a symptom of a disorder.

As used herein, the term “elevated heparan sulfate” refers to a subject having serum heparan sulfate concentrations at or above the baseline concentrations of the subject. An example of “elevated heparan sulfate” includes, but is not limited to a serum concentration of about 6.5 μg/mL to about 15 μg/mL, or about 6.5 μg/mL to about 14 μg/mL, or about 6.5 μg/mL to about 13 μg/mL, or about 6.5 μg/mL to about 12 μg/mL, or about 6.5 μg/mL to about 11 μg/mL, or about 6.5 μg/mL to about 12 μg/mL, or about 6.5 μg/mL to about 10 μg/mL, or about 6.5 μg/mL to about 9 μg/mL, or about 7 μg/mL to about 14 μg/mL, or about 7.5 μg/mL to about 14 μg/mL, or about 7.5 μg/mL to about 13 μg/mL, or about 8 μg/mL to about 13 μg/mL, or about 8.5 μg/mL to about 14 μg/mL, or about 8 μg/mL to about 14 μg/mL, or about 9 μg/mL to about 13 μg/mL, or about 9 μg/mL to about 14 μg/mL, or about 9 μg/mL to about 15 μg/mL, or about 10 μg/mL to about 15 μg/mL, or about 11 μg/mL to about 15 μg/mL, or about 12 μg/mL to about 15 μg/mL, or about 13 μg/mL to about 15 μg/mL, or about 14 μg/mL to about 15 μg/mL, which indicates grade I-II GVHD, or mild GVHD, or a serum heparan sulfate concentration of about 15.5 μg/mL to about 30 μg/mL, or about 16 μg/mL to about 29 μg/mL, or about 16 μg/mL to about 27 μg/mL, or about 16 μg/mL to about 25 μg/mL, or about 17 μg/mL to about 30 μg/mL, or about 18 μg/mL to about 30 μg/mL, or about 18 μg/mL to about 27 μg/mL, or about 18 μg/mL to about 25 μg/mL, or about 18 μg/mL to about 23 μg/mL, or about 18 μg/mL to about 20 μg/mL, or about 20 μg/mL to about 23 μg/mL, or about 23 μg/mL to about 25 μg/mL, or about 25 μg/mL to about 30 μg/mL, or greater than 30 μg/mL, which indicates diseases including grade III-IV GVHD, or severe GVHD. Another example of “elevated heparan sulfate” includes, but is not limited to a serum concentration of about 10 μg/mL to about 40 μg/mL, or about 20 μg/mL to about 40 μg/mL, or about 30 μg/mL to about 40 μg/mL, or greater than 10 μg/mL, or greater than 20 μg/mL, or greater than 30 μg/mL, or greater than 40 μg/mL, which indicates the onset of acute cardiac allograft rejection in the subject.

As used herein, the term “prevention” means generally the prevention of the establishment of an immune-mediated injury caused by elevated levels of serum heparan sulfate. Prevention may be primary, secondary or tertiary. For example, primary prevention refers to the prevention of the establishment of the disease. Secondary prevention refers to intervention in subjects who are at high risk for the development of an immune-mediated injury caused by elevated levels of serum heparan sulfate but have not yet developed the disease. These subjects may or may not have exhibited some physiological symptoms. Tertiary prevention refers to preventing the worsening of the immune-mediated injury caused by elevated levels of serum heparan sulfate, and reducing the symptoms experienced by the subjects. An example of prevention includes, but is not limited to, a subject that maintains serum heparan sulfate concentration of less than 2 μg/mL, or about 2 μg/mL to about 6 μg/mL, or about 2 μg/mL to about 5.5 μg/mL, or about 2 μg/mL to about 5 μg/mL, or about 2 μg/mL to about 4.5 μg/mL, or about 2 μg/mL to about 4 μg/mL, or about 3 μg/mL to about 6 μg/mL, or about 3 μg/mL to about 6 μg/mL, or about 3 μg/mL to about 5.5 μg/mL, or about 3 μg/mL to about 5 μg/mL, or about 3 μg/mL to about 4.5 μg/mL, or about 3 μg/mL to about 4 μg/mL, or about 3.5 μg/mL to about 6 μg/mL, or about 3.5 μg/mL to about 6 μg/mL, or about 3.5 μg/mL to about 5.5 μg/mL, or about 3.5 μg/mL to about 5 μg/mL, or about 3.5 μg/mL to about 4.5 μg/mL, or about 3.5 μg/mL to about 4 μg/mL, or about 3 μg/mL, or about 3.5 μg/mL, or about 4 μg/mL, or about 4.5 μg/mL, which indicates a grade 0 GVHD, or no evidence of disease, following a cellular transplant, including but not limited to, allogeneic hematopoietic stem cell transplantation. Another example of prevention includes, but is not limited to, a subject that maintains a serum heparan sulfate concentration of less than 10 μg/mL following cardiac allograft transplant.

In one embodiment, the heparan sulfate inhibitor is a serine protease inhibitor. Examples of a serine protease inhibitor include, but are not limited to, 4-(2-Aminoethyl)benzenesulfonyl fluoride hydrochloride, α1-antitrypsin, α2-antitrypsin, antithrombin, C1-inhibtior, camostat, maspin, methoxy arachidonyl fluorophosphonate, plasminogen activator inhibitor-1, Plasminogen activator inhibitor-2 , phenylmethylsulfonyl fluoride, protein C inhibitor, and protein-z related inhibitor. In certain embodiments, the serine protease inhibitor is α1-antitrypsin.

In one embodiment, the heparan sulfate inhibitor is an enzyme that degrade heparan sulfate. An example of such enzyme includes, but is not limited to, heparanase.

In one embodiment, the organ transplant is a solid organ transplant. Examples of a solid organ transplant include, but are not limited to, heart, kidney, liver, lung, pancreas, and intestine. In certain embodiments, solid organ transplant include heart and kidney. In certain embodiments, solid organ transplant include heart. In another embodiment, the organ transplant is a tissue transplant. Examples of a tissue transplant include, but are not limited to, bone, tendon, cornea, skin, heart valve, and veins. In yet another embodiment, the organ transplant is a cellular transplant. Examples of a cellular transplant include, but are not limited to, stem cells, bone marrow, abdominal, and pancreases islet cells. In a certain embodiment, the stem cells are allogeneic hematopoietic stem cells.

As used herein, the term “administration” or “administering,” refers to providing, contacting, a compound or compounds by any appropriate route to achieve the desired effect. In certain embodiments, the term “administration” may also include the delivery of a compound, such as a heparan sulfate inhibitor. These compounds may be administered to a subject in numerous ways including, but not limited to, oral, sublingual, parenteral (e.g., intravenous, subcutaneous, intracutaneous, intramuscular, intraarticular, intraarterial, intrasynovial, intrasternal, intrathecal, intralesional or intracranial injection), transdermal, topical, buccal, rectal, vaginal, nasal, ophthalmic, via inhalation, and implants.

When formulating the pharmaceutical compositions described herein, the clinician may utilize preferred dosages as warranted by the condition of the subject being treated.

The actual dosage of the heparan sulfate inhibitor and/or any additional immunosuppressant agent or conditioning regimen employed may be varied depending upon the requirements of the subject and the severity of the condition being treated. Determination of the proper dosage for a particular situation is within the skill of the art. Generally, treatment is initiated with smaller dosages which are less than the optimum dose of the compound. Thereafter, the dosage is increased by small amounts until the optimum effect under the circumstances is reached.

In some embodiments, when a heparan sulfate inhibitor is administered in combination with one or more additional immunosuppressant agents, the additional immunosuppressant agent (or agents) is administered at a standard dose. Examples of immunosuppressant agents include, but are not limited to, corticosteroids, calcineurin inhibitors, an anti-proliferative agent, and m-TOR inhibitors. Examples of corticosteroids used as immunosuppressant agents include, but are not limited to, prednisolone and hydrocortisone. Examples of calcineurin inhibitors used as immunosuppressant agents include, but are not limited to, ciclosporin and tacrolimus. Examples of anti-proliferative agents used as immunosuppressant agents include, but are not limited to, azathioprine and mycophenolic acid. Examples of mTOR inhibitors include, but are not limited to, sirolimus and everolimus. In certain embodiments, the immunosuppressant agent comprises cellcep, calcineurin inhibitor, prednisone, and sirolimus.

In other embodiments, when a heparan sulfate inhibitor is administered in combination with one or more additional conditioning regimens, the additional conditioning regimen (or regimens) is administered at a standard dose. Examples of conditioning regimens include, but are not limited to, chemotherapy, monoclonal antibody therapy, total body irradiation, ablative, and non-ablative/reduced intensity. In one embodiment, the conditioning regimen comprises ablative, non-ablative/reduced intensity, or total body irradiation.

In accordance with experience and knowledge, the practicing physician can modify each protocol for the administration of a component (heparan sulfate inhibitor and immunosuppressant compositions and/or conditioning regimen) of the treatment according to the individual subject's needs, as the treatment proceeds. The attending clinician, in judging whether treatment is effective at the dosage administered, will consider the general well-being of the subject as well as more definite signs such as relief of disease-related symptoms. Relief of disease-related symptoms such as pain, and improvement in overall condition can also be used to help judge effectiveness of treatment.

In one embodiment, a method of diagnosing an injurious condition that is associated with elevated heparan sulfate in a subject that was the recipient of a transplanted organ, tissue, or cells, by collecting a biological sample from the subject and determining the serum concentration of heparan sulfate, where the concentration of heparan sulfate directly correlates with the severity of the heparan sulfate-mediated immune injury. In alternative embodiments a method of diagnosing further comprises administering a therapeutically effective amount of an inhibitor, including heparin sulfate. An additional embodiment, a method of diagnosis comprises wherein the heparan sulfate-mediated immune injury is graft-versus-host disease, and where the severity of the graft-versus-host disease is determined by a serum heparan sulfate concentration. A serum heparan sulfate concentration of less than 2 μg/mL, or about 2 μg/mL to about 6 μg/mL , or about 2 μg/mL to about 5.5 μg/mL, or about 2 μg/mL to about 5 μg/mL, or about 2 μg/mL to about 4.5 μg/mL, or about 2 μg/mL to about 4 μg/mL, or about 3 μg/mL to about 6 μg/mL, or about 3 μg/mL to about 6 μg/mL, or about 3 μg/mL to about 5.5 μg/mL, or about 3 μg/mL to about 5 μg/mL, or about 3 μg/mL to about 4.5 μg/mL, or about 3 μg/mL to about 4 μg/mL, or about 3.5 μg/mL to about 6 μg/mL, or about 3.5 μg/mL to about 6 μg/mL, or about 3.5 μg/mL to about 5.5 μg/mL, or about 3.5 μg/mL to about 5 μg/mL, or about 3.5 μg/mL to about 4.5 μg/mL, or about 3.5 μg/mL to about 4 μg/mL, or about 3 μg/mL, or about 3.5 μg/mL, or about 4 μg/mL, or about 4.5 μg/mL indicates a grade 0 GVHD, or no evidence of disease. A serum heparan sulfate concentration of about 6.5 μg/mL to about 15 μg/m, or about 6.5 μg/mL to about 14 μg/mL, or about 6.5 μg/mL to about 13 μg/mL, or about 6.5 μg/mL to about 12 μg/mL, or about 6.5 μg/mL to about 11 μg/mL, or about 6.5 μg/mL to about 12 μg/mL, or about 6.5 μg/mL to about 10 μg/mL, or about 6.5 μg/mL to about 9 μg/mL, or about 7 μg/mL to about 14 μg/mL, or about 7.5 μg/mL to about 14 μg/mL, or about 7.5 μg/mL to about 13 μg/mL, or about 8 μg/mL to about 13 μg/mL, or about 8.5 μg/mL to about 14 μg/mL, or about 8 μg/mL to about 14 μg/mL, or about 9 μg/mL to about 13 μg/mL, or about 9 μg/mL to about 14 μg/mL, or about 9 μg/mL to about 15 μg/mL, or about 10 μg/mL to about 15 μg/mL, or about 11 μg/mL to about 15 μg/mL, or about 12 μg/mL to about 15 μg/mL, or about 13 μg/mL to about 15 μg/mL, or about 14 μg/mL to about 15 μg/mL, indicates grade I-II GVHD, or mild GVHD. A serum heparan sulfate concentration of about 15.5 μg/mL to about 30 μg/mL, or about 16 μg/mL to about 29 μg/mL, or about 16 μg/mL to about 27 μg/mL, or about 16 μg/mL to about 25 μg/mL, or about 17 μg/mL to about 30 μg/mL, or about 18 μg/mL to about 30 μg/mL, or about 18 μg/mL to about 27 μg/mL, or about 18 μg/mL to about 25 μg/mL, or about 18 μg/mL to about 23 μg/mL, or about 18 μg/mL to about 20 μg/mL, or about 20 μg/mL to about 23 μg/mL, or about 23 μg/mL to about 25 μg/mL, or about 25 μg/mL to about 30 μg/mL, or greater than 30 μg/mL indicates grade III-IV GVHD, or severe GVHD. In yet other embodiments, where the heparan sulfate-mediated immune injury is acute cardiac allograft rejection, a serum heparan sulfate concentration of about 10 μg/mL to 40 μg/mL, or about 20 μg/mL to about 40 μg/mL, or about 30 μg/mL to about 40 μg/mL, or greater than 10 μg/mL, or greater than 20 μg/mL, or greater than 30 μg/mL, or greater than 40 μg/mL indicates the onset of acute cardiac allograft rejection.

In one embodiment, a biological sample is collected and assayed from a subject to determine the serum concentration of heparan sulfate. Examples of a biological sample include, but are not limited to, blood and plasma.

In one embodiment, a biological sample is collected from the subject 7-100 days following allogeneic hematopoietic stem cell transplantation. In another embodiment, a biological sample is collected from the subject 14-100 days following allogeneic hematopoietic stem cell transplantation. In certain embodiments, a biological sample is collected from the subject 30-100 days following allogeneic hematopoietic stem cell transplantation. In certain embodiments, a biological sample is collected from the subject 60-100 days following hematopoietic stem cell transplantation.

The following examples are offered by way of illustration and not by way of limitation.

### EXAMPLES

### Example 1

### HS Promotes Alloreactive T Cell Proliferation by Stimulating TLR4 on Dendritic Cells

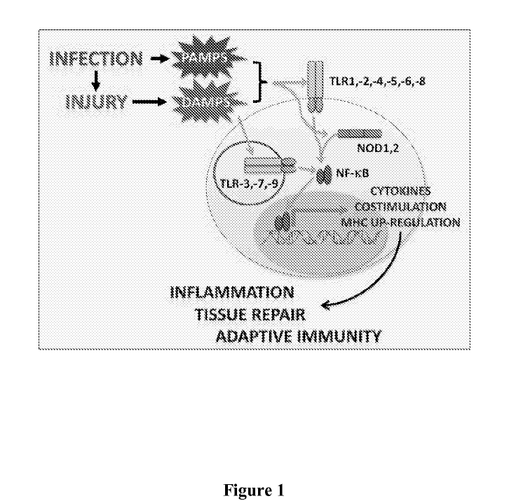

To identify endogenous innate immune activators with significant contribution to the alloimmune response, various DAMPs that have been implicated in stimulating TLR pathways in promoting alloreactive T cell responses were examined. (FIG. 1).

Mice: BALB/c mice were purchased from the National Cancer Institute (Frederick, Md., USA). B10.D2 and TLR4^{-/-} BALB/c mice were purchased from The Jackson Laboratory (Bar Harbor, Me., USA), respectively. MyD88^{-/-} mice were kindly provided by Dr. Shizuo Akira (Osaka University, Osaka, Japan) and have been backcrossed for greater than 10 generations onto the BALB/c background. Donor mice were males between 8 and 12 weeks of age and recipient mice were males between 12 and 16 weeks of age (˜22-26 grams). All experimental procedures involving the use of mice were done in accordance with protocols approved by the Animal Care and Use Committee of Duke University.

Reagents and cell lines: The following reagents were used: LPS from Escherichia coli O111:B4 (List Biological Laboratories, Inc., Campbell, Calif., USA), endotoxin-free Pam3CSK4 (InvivoGen, San Diego, United States), bovine kidney heparan sulfate (Seikagaku, Tokyo, Japan), Hyaluronan Select HA 150K (Sigma, St. Louis, United States; S-0201), fibronectin (Sigma; F-2006), fibrinogen (Hyphen BioMed, Neuville-sur-Oise, France), HMGB1 (Abnova, Taipei City, Taiwan), Hsp70 (Assay Designs, Ann Arbor Mich., USA), C12 IE-DAP and L18-MDP (InvivoGen), protamine sulfate (Sigma), and murine GM-CSF (R&D Systems, Minneapolis, Minn., USA). Sonicated hyaluronan, a gift from Dr. Stavros Garantziotis (NIEHS, Research Triangle Park, United States) was produced as previously described. (Garantziotis, S. et al. (2009) J. Biol. Chem. 17:11309-17).

HEK 293 cell lines co-expressing human CD14, MD2 and TLR4 or TLR2 were a kind gift from Dr. Michael Fessler (NIEHS, Research Triangle Park, N.C., USA). Some assays were performed in the presence of polymyxin B (Sigma, St. Louis, United States) or after pre-incubation with heparinase III from Flavobacterium heparinum (Sigma, St. Louis, United States). One unit heparinase was incubated with 50 μg of HS or 50 ng LPS in 25 μL culture medium at 32° C. for 6 hr.

T cell proliferation assay: DCs were generated from bone marrow as previously described. (Yang, Y. (2004) Nat. Immunol. 5:508-15). CD3⁺ T cells were isolated from B6 mice using a CD3 negative selection magnetic bead kit (Invitrogen, Carlsbad, United States). Proliferation assays were performed as previously described. (Brennan, T. V. et al. (2008) Transplantation 85:247-55).

Statistical analysis: Results were expressed as mean+/−SEM. Comparison between groups was performed by Kruskal-Wallis and Student t-test for continuous variables, Fisher's exact test for categorical variables, and log rank test for survival data. All statistical analyses were performed using Prism v5.0 software (GraphPad Software, La Jolla, United States). Differences were reported to be significant with p≦0.05.

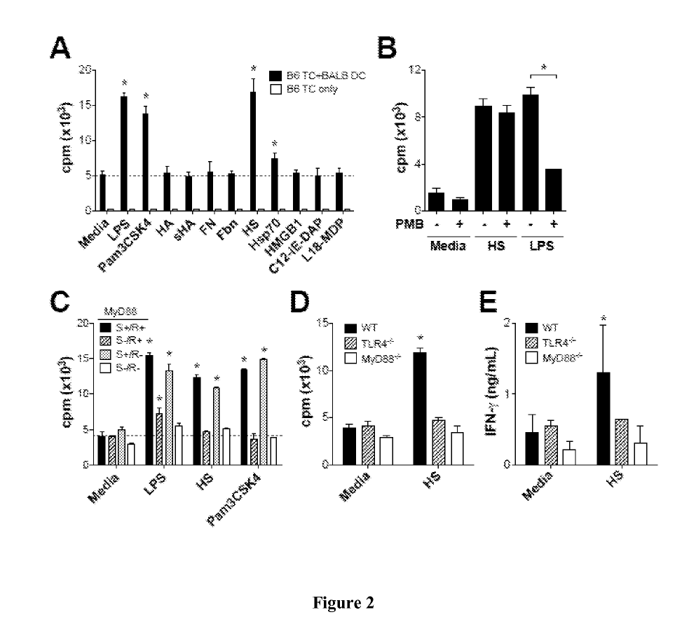

Purified T cells from C57BL/6 mice were stimulated with bone marrow-derived DCs from BALB/c mice and their proliferation was measured by the incorporation of ³H-thymidine. DAMPs tested included proteins—fibronectin (FN), fibrinogen (Fbn), heat shock protein 70 (HSP70), and high-mobility group protein B1 (HMGB1); and glucosaminoglycans—heparan sulfate (HS) and hyaluronan (HA). For comparison, two well-described PAMPs, lipopolysaccharide (LPS) and a synthetic tripalmitoylated lipopeptide (Pam3CSK4) that are ligands of the TLR4 homodimer and the TLR1/2 heterodimer, respectively, were tested. As nucleotide-binding domain, leucine-rich repeat containing receptors (NLRB) are another family of innate immune receptors, ligands of NLR1 (C12-iE-DAP) and NLR2 (L18-MDP) were further tested. Among DAMPs, only HS and HSP70 significantly increased alloreactive T cell proliferation compared to media alone (FIG. 2A). The stimulation by HS was comparable to that achieved by PAMPs such as LPS and Pam3CSK4. Neither of the NLR ligands tested produced a significant increase in alloreactive T cell proliferation.

To exclude the possibility of LPS contamination as a cause for the stimulatory effect of HS, the proliferation assay was performed with HS in the presence or absence of the LPS inhibitor, polymyxin B (PMB) (FIG. 2B). PMB caused a significant decrease in LPS-induced proliferation, but not in HS-induced proliferation, indicating that LPS contamination was not responsible for the increase in responder T cell proliferation observed with HS treatment.

Next, to investigate whether HS enhanced proliferation of allogeneic T cells was due to the stimulation of TLRs on DCs or T cells, because MyD88 is a common adaptor protein involved in the signal transduction of all TLRs with the exception of TLR3⁹, HS was tested against DCs or T cells that were deficient for MyD88. For this purpose, T cells from WT or MyD88^{-/-} C57BL/6 mice were stimulated with DCs from WT or MyD88^{-/-} BALB/c mice in an allogeneic proliferation assay in the presence of HS, LPS, Pam3CSK4, or media alone. As shown in FIG. 2C, the lack of MyD88 in DCs, but not in allogeneic T cells, abolished the enhanced proliferation by HS, which was similar to those found with LPS and Pam3CSK4, with the exception that LPS still produced a reduced, but significant increase in proliferation with MyD88^{-/-} DCs as stimulators. These results suggest that HS stimulates TLRs on DCs, but not T cells, to enhance T cell proliferation.

HS has been shown as a TLR4 ligand, (Johnson, G. B. et al., (2002) J. Immunol. 168:5233-39; Johnson, G. B., Brunn, G. J. & Platt, J. L. (2004) J. Immunol. 172:20-24). To determine whether the absence of TLR4 expression on stimulating DCs would reduce HS-induced alloreactive T cell proliferation equivalent to MyD88 deficiency, purified T cells from C57BL/6 mice were co-cultured with irradiated WT, TLR4^{-/-}, and MyD88^{-/-} BALB/c DCs and proliferation was measured by the incorporation of ³H-thymidine. Indeed, HS was not able to increase the proliferation of allogeneic T cells above baseline (FIG. 2D) or increase IFN-γ production (FIG. 2E) when TLR4^{-/-} DCs were used, similar to when MyD88^{-/-} DCs were used. These results indicate that an intact TLR4-MyD88 pathway in DCs, but not in T cells, is necessary for HS to promote the alloreactive T cell response.

### Example 2

### HS Stimulates TLR4-Dependent DC Maturation and Function

To investigate how activation of DCs by HS promoted the alloreactive T cell response, an as DC maturation and production of pro-inflammatory cytokines are key initial events in triggering adaptive immune responses, the ability of HS to upregulate DC expression of costimulatory molecules, CD40 and CD80, and pro-inflammatory cytokines, IL-6 and IL-12 was tested.

Antibodies and flow cytometry: Anti-CD40 (HM40-3), anti-CD80 (16-10A1), anti-Thy1.1 (OX-7), anti-Ly5.1 (A20) anti-IFN-γ (XMG1.2), rat IgG1 isotype (R3-34), and the BrdU Flow Kit (FITC-labeled) were from BD Biosciences (San Jose, Calif., USA). Intracellular IFN-γ staining was performed as previously described. (Brennan, T. V. et al. (2008) Transplantation 85:247-55). For in vivo BrdU labeling, mice were injected with 50 μg BrdU/gm i.p. 1 hour prior to analysis. Collection of flow cytometric data was acquired using a FACSCanto (BD Biosciences), and events were analyzed using FloJo software (Tree Star, Inc., Ashland, United States).

Cytokine analysis: Cell culture supernatants were obtained from DC cultures or T cell proliferation assays and assayed for IL-6, IL-12 and IFN-γ by ELISA (BD Biosciences) according to the manufacturer's standard protocols. HEK 293 supernatants were tested for human IL-8 by ELISA (BioLegend, San Diego, United States).

Luciferase reporter assay: Luciferase activity was measured using the Dual-Luciferase Reporter Assay (Promega, Madison, Wis., USA) according to the manufacture's recommended protocol. Luminescence was measured using an LMax Luminometer (Molecular Devices, Sunnyvale, United States).

HS ELISA: Serum samples were assayed for HS concentration by ELISA (Amsbio LLC, Lake Forest, Untied States) according to the manufacturer's recommended protocol. HS levels were also measured in patients undergoing Allo-HSCT under an institution-sponsored IRB (Pro00031607). Patient data were obtained by chart review. Serum samples were collected from patients at various time points relative to GVHD and assayed for HS levels by ELISA as described above.

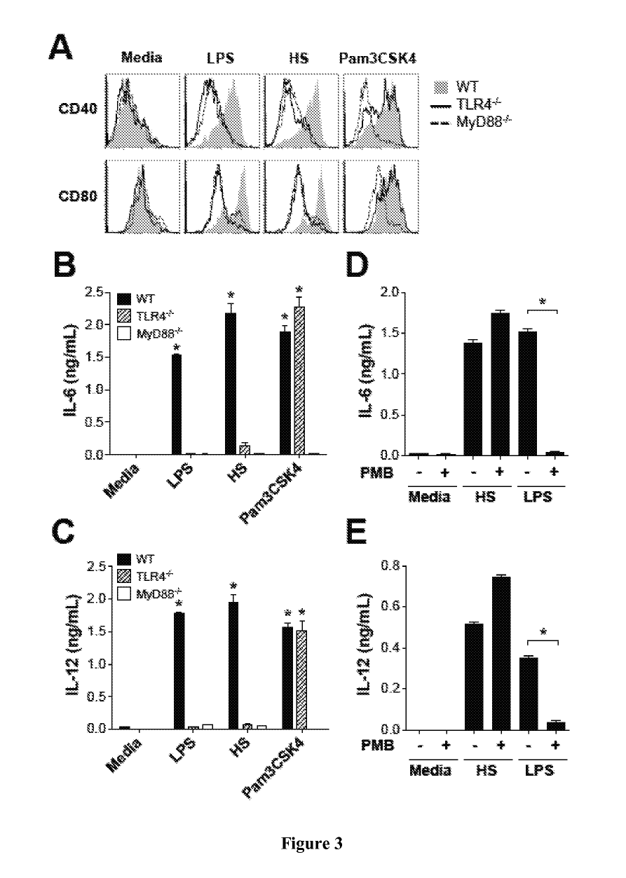

Similar to LPS, HS caused the upregulation of CD40 and CD80 on WT DCs, but not on TLR4^{-/-} or MyD88^{-/-} DCs (FIG. 3A). In comparison, Pam3CSK4 (TLR1/2 ligand) was able to upregulate CD40 and CD80 on WT and TLR4^{-/-} DCs, but not on MyD88^{-/-} DCs. Similarly, HS stimulated DCs to produce pro-inflammatory cytokines, IL-6 and IL-12, from WT DCs, but not from TLR4^{-/-} or MyD88^{-/-} DCs (FIGS. 3B & 3C) that was not inhibited by PMB (FIGS. 3D & 3E).

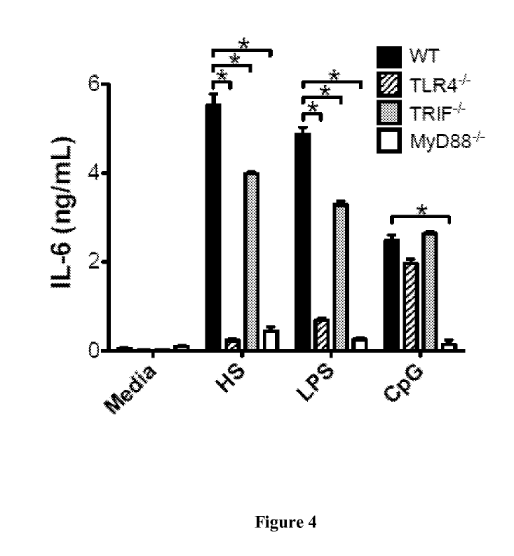

TRIF is a well-described intracellular signal transduction adaptor molecule involved in MyD88-independent TLR4 signal transduction. (Yamamoto, M. et al. (2003) Science 301:640-43). Therefore, HS stimulation of IL-6 production from WT, TLR4^{-/31} , MyD88^{-/-} and TRIF^{-/-} C57BL/6 DCs was tested. While MyD88-deficiency prevented the majority of the HS-induced IL-6 production, TRIF deficiency had a smaller, but significant effect on the production of IL-6 upon stimulation with HS and LPS, but not CpG (FIG. 4).

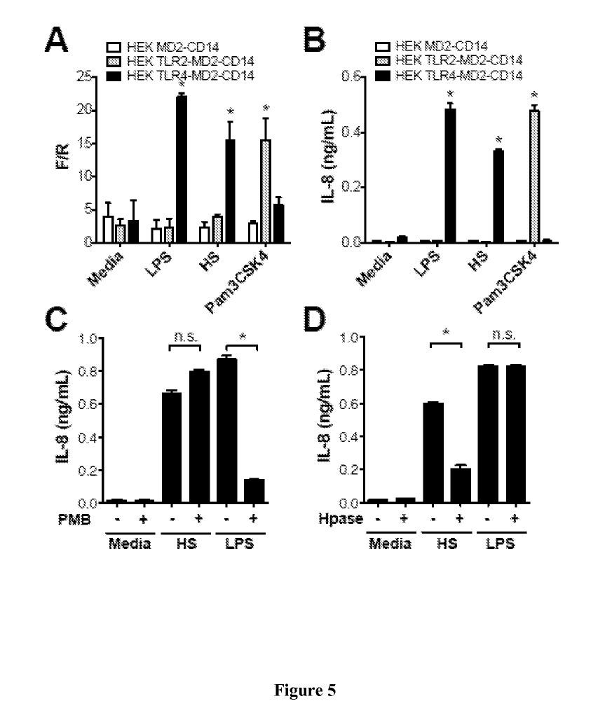

A heterodimer of different TLRs is required for some PAMPs. Thus, to determine whether if TLR4 was sufficient for HS activity, the ability of HS to activate human epithelial kidney (HEK) cell lines stably transfected to express human TLR4 or human TLR2 in addition to the co-receptors CD14 and MD2 was tested. These cell lines were transfected with a NF-κB promoter driven firefly luciferase reporter plasmid along with a thymidine kinase (TK) promoter driven Renilla luciferase reporter plasmid as a transfection control. The ratio of luminescence produced by firefly luciferase to Renilla luciferase (F/R) was then measured in response to media alone, LPS, HS, and Pam3CSK4. The production of IL-8, which is downstream of NF-κB activation, by ELISA was also measured. Similar to LPS, we found that HS caused NF-κB activation (FIG. 5A), as well as IL-8 expression (FIG. 5B) in TLR4 expressing cell lines. Thus, TLR4 is sufficient for HS-induced NF-κB activation and IL-8 production.

To control for potential LPS contamination, PMB was added to the cultures. Treatment with PMB inhibited IL-8 expression resulting from LPS treatment, but not HS treatment (FIG. 5C). To demonstrate a direct role of HS, HS pre-treated with heparanase was tested next. Heparanase significantly reduced HS stimulation, but not LPS stimulation, demonstrating that HS induced IL-8 expression was specific to HS (FIG. 5D).

### Example 3

### Serum Levels of HS are Elevated at the Onset of GVHD in a Murine Model of Allo-HSCT

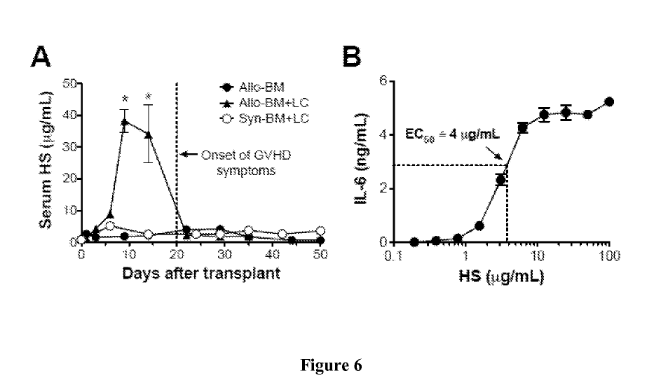

To investigate the in vivo relevance of HS in the setting of alloimmunity, serum levels of HS were tested in a mouse model of GVHD in the setting of Allo-HSCT (FIG. 6A).

Allo-HSCT: For Allo-HSCT, wild-type (WT) and TLR4−/− BALB/c mice received myeloablative total-body irradiation (8.5 Gy) followed by intravenous infusion of 1×10⁷ B10.D2 or BALB/c T-cell depleted bone marrow (TCD-BM). Bone marrow was prepared as previously (Yang, Y. et al. (2004) Nat. Immunol.5:508-15) and the T cells were depleted using Thy1.2 (Invitrogen, Carlsbad, United States) or CD5 (Miltenyi Biotec, Auburn, United States) conjugated magnetic beads according to the manufactures' instructions. To induce GVHD, 5×10⁶ lymph node cells from inguinal, axillary, cervical and mesenteric lymph nodes of B10.D2 or BALB/c mice were injected intravenously in addition to TCD-BM.

In another model of acute GVHD, 5×10⁶ lymph node cells and 1×10⁷ TCD BM from C3H.SW were transferred to C57BL/6 recipients irradiated with 10 Gy. HSCT recipients of these two GVHD models then received either intraperitoneal (i.p.) injections of 2 mg of al-antitrypsin (A1AT, Aralast™, Baxter, Deerfield, United States) resuspended in 200 μL sterile phosphate-buffered saline (PBS), subcutaneous (s.c.) injections of 20 mg/kg PG545 (Progen Pharmaceuticals Limited, Queensland, Australia) in 200 μL PBS, or 200 μL PBS alone at the indicated intervals.

In this model, lethally irradiated BALB/c mice were transplanted with 1×10⁷ T cell-depleted bone marrow (TCD-BM) and 5×10⁶ lymphocytes (LCs) from B10.D2 mice (Allo-BM+LC). Control mice received B10.D2 TCD-BM only (Allo-BM only) or syngeneic BALB/c TCD-BM and 5×10⁶ BALB/c LCs (Syn-BM+LC). Serum HS levels were significantly elevated in the recipients of Allo-BM+LC on post-transplant days 9 and 14, and returned to baseline by post-transplant day 22. Notably, the increase in HS occurred prior to the onset of GVHD symptoms.

To determine if the in vivo concentrations of HS were sufficient to cause DC activation, the production of IL-6 by cultured DCs in response to a range of HS concentrations was tested (FIG. 6B). Under these conditions, maximal response was achieved by HS concentrations of 12.5 μg/mL and that the half maximal effective concentration (EC₅₀) of HS was approximately 4 μg/mL.

### Example 4

### A1AT Reduces Serum HS Levels and Improves the Outcome of GVHD in a TLR4-Dependent Manner

The significance of HS elevation in the development of GVHD was examined A1AT is a potent serum protease inhibitor used in patients with al-antitypsin deficiency to protect against neutrophil elastase-induced lung injury. (Silverman, E. K. & Sanhaus R. A. (2009) N. Engl. J. Med. 360:2749-57). It has been previously shown that intravenous treatment of mice with elastase, a protease that cleaves HS-containing proteoglycans within the extracellular matrix, causes a systemic inflammatory response syndrome that is similar to that which occurs when HS is injected directly. (Johnson, G. B., Brunn, G. J. & Platt, J. L. (2004) J. Immunol. 172:20-24).

Assessment of GVHD: GVHD severity was assessed using the previously described clinical scoring system that accounts for five parameters: weight loss, fur texture, skin integrity, hunching posture, and activity. (Cooke, K. R. et al. (1996) Blood 88:3230-39). Endpoints for survival were death, moribund status, or weight loss >30%. Histologic analysis of GVHD was performed on full-thickness ear tissue. Following fixation in fresh neutral buffered formalin for 24 hours, ear tissue was routinely processed, embedded in paraffin and the 5 μm thick sections were stained with hematoxylin and eosin. These de-identified slides were evaluated by a single pathologist (DMC) blinded to experimental groups and graded in a semi-quantitative fashion on the basis of dermal fibrosis, fat loss, inflammation, epidermal interface changes, and follicular drop-out (0-2 for each category). (Anderson, B. E. et al (2003) J. Clin. Invest. 112:101-108).

Thus, to determine whether the administration of A1AT would reduce serum HS levels following Allo-HSCT, A1AT (2 mg) was administered to Allo-BM+LC recipients by i.p. injection every 3 days beginning one day prior to transplant, as described previously for the use of A1AT therapy for tolerance induction in the setting of pancreatic islet transplantation. (Lewis, E. C. et al. (2008) Proc. Nat'l. Acad. Sci. U.S.A. 105:16236-41).

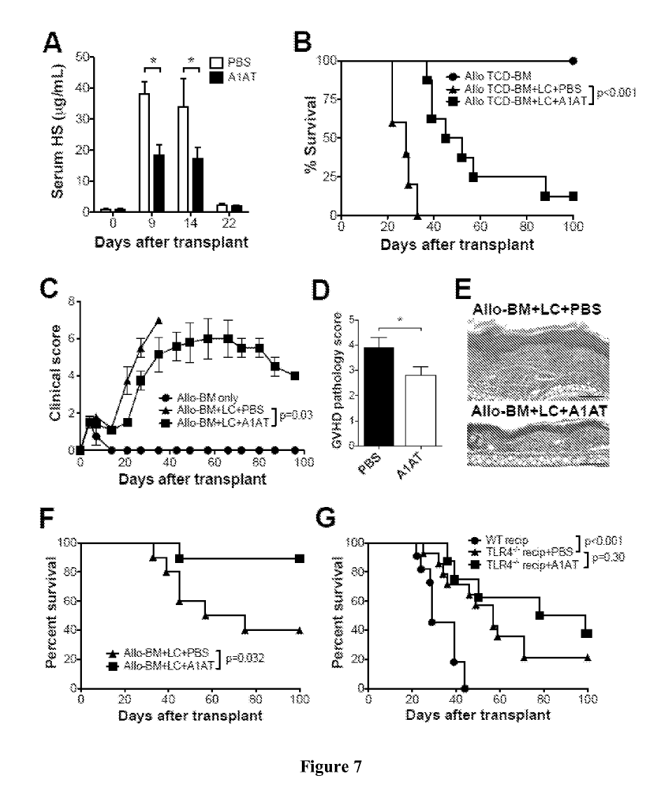

Compared to injections of PBS alone, A1AT-treated recipients had significantly lower serum HS levels at days 9 and 14 following HSCT (FIG. 7A). In the comparison of survival between the A1AT treated and PBS treated recipients, A1AT therapy resulted in significantly longer survival compared to PBS treated controls (MST 48.5 vs. 28.0 days; p<0.001) (FIG. 7B).

Clinical scores for GVHD were compared between experimental groups using a 10-point scale. (Cooke, K. R. et al. (1996) Blood 88:3230-3239). All groups had an elevation in clinical score between 3-7 days after transplantation related to radiation exposure (FIG. 7C). The elevation in clinical score quickly returned to the baseline thereafter in the cohort that received allogeneic TCD-BM alone. However, the cohort that received the allogeneic TCD-BM and LCs (Allo-BM+LC) had a progressive increase in clinical score beginning 20 days after transplantation until they reached their clinical endpoint (death or 30% wt loss). Allo-BM+LC recipients treated with A1AT had a significantly lower clinical score curve (p=0.03).

Severity of GVHD was also examined by histology. Ear skin was obtained from Allo-BM+LC mice at 3 weeks post-transplant from A1AT or PBS treated recipients and scored for GVHD severity. A1AT treated mice demonstrated significantly lower GVHD pathology scores (FIGS. 7D-E).

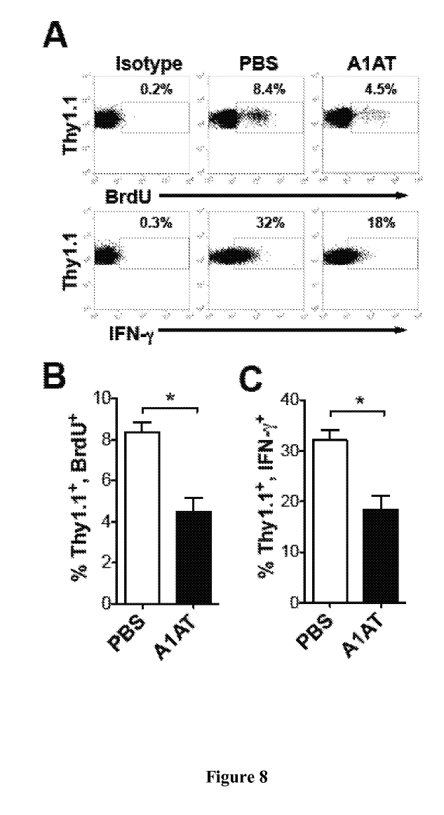

To investigate whether the improved survival by A1AT administration in the setting of Allo-HSCT may be due to the suppression of alloreactive T cell responses, donor T cells were tested for in vivo proliferation by BrdU incorporation and for function by IFN-γ production following Allo-HSCT. To monitor the behavior of alloreactive T cells in vivo, allogeneic TCD-BM and LCs isolated from Thy1.1⁺ B10.D2 donor mice were utilized. Recipients were pulsed with BrdU six days after transplantation and their splenocytes were FACS analyzed one hour later. BrdU incorporation and IFN-γ production by donor Thy1.1⁺ T cells were significantly reduced in recipient mice treated with A1AT (FIGS. 8A-C).

To further support the survival benefit obtained from A1AT therapy, a second GVHD model was performed. Lethally irradiated C57BL/6 recipients were transplanted with 10⁷ C3H.SW TCD-BM and 5×10⁶ C3H.SW LCs. One group received A1AT injection as described above and the other received PBS control injection. Again, A1AT treated recipients demonstrated a significant survival benefit compared with the PBS treated group (p=0.032) (FIG. 7F).

Based on our in vitro results, the contribution of HS towards allospecific T cell activation is TLR4 dependent. The survival of WT BALB/c recipients of B10.D2 TCD BM and LCs was compared to TLR4^{-/-} BALB/c recipients that either received A1AT injections or PBS control injection. Compared to WT BALB/c recipients, TLR4^{-/-} recipients had a significantly longer survival (57 vs. 29 days, p<0.001). However, no further survival benefit was observed in TLR4^{-/-} recipients treated with A1ATcompared to the PBS treated TLR4^{-/-} mice (p=0.30, FIG. 7G), suggesting the effect of A1AT in vivo is also dependent on TLR4.

### Example 5

### The HS mimetic, PG545, Increases Serum HS Levels and Exacerbates GVHD Following Allo-HSCT

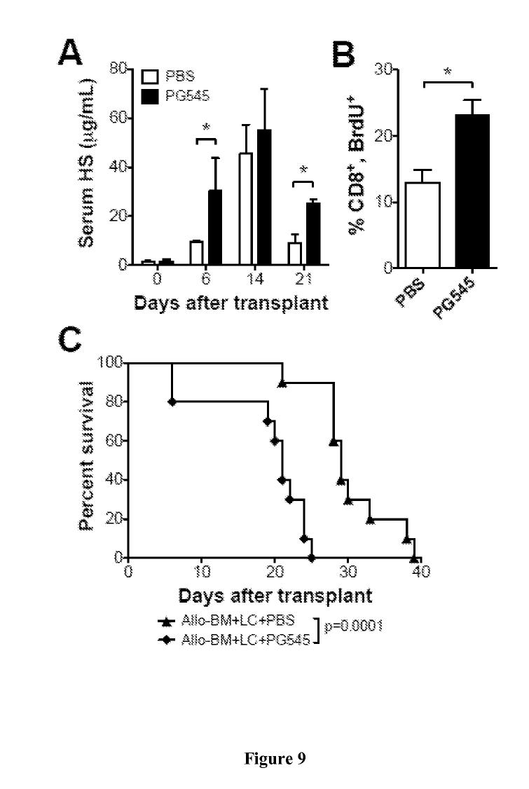

To determine whether increasing serum levels of HS could increase the alloreactive T cell response and accelerate GVHD, 20 mg/kg of PG545 was administered once per week by subcutaneous injection beginning one day prior to transplant. For this purpose the HS mimetic, PG545 (Progen Pharmaceuticals, Queensland, Australia), which may function as a competitive inhibitor of heparanase was used. (Dredge, K. et al. (2011) Br. J. Cancer 104:635-42). PG545 therapy resulted in higher post-transplant serum HS levels (FIG. 9A). Analysis of BrdU uptake on post-transplant day 6 revealed that a higher percentage of proliferating donor CD8 T cells in the PG545 treated group compared to the PBS treated group (23.2±2.3 vs. 12.9±2.0; n=3 per group; p<0.05) (FIG. 9B). PG545 treatment also accelerated the rate of GVHD compared with PBS control injections (21 vs. 29 days, p<0.001) (FIG. 9C). These data demonstrate that HS can modulate the alloreactive T cell response in vivo.

### Example 6

### Recipient Antigen Presenting Cells are Present at Time of HS Elevation

To demonstrate the persistence of recipient DCs at the time of HS elevation, B6→BALB/c HSCT model of GVHD consisting of lethal irradiation and the transfer of 10⁷ TCD-BM+10⁶ LCs was used.

T cell receptor transgenic (TCR-tg) adoptive transfer: Lymph node (LN) cells purified from 4C-TCR-tg mice on the C57BL/6-Ly5.1 background were labeled with carboxy fluorescein succinimidyl ester (CFSE; Life Technologies, Grand Island, United States) as previously described. (Brennan, T. V. et al. (2008) Transplantation 85:247-55). BALB/c recipients of C57BL/6 HSCTs (10⁷ TCD-057BL/6 BM and 106 C57BL/6 LN cells) were intravenously injected with 2×10⁶ CFSE-labeled LN cells 14 days after transplant. Three days later, mice were sacrificed, their livers were harvested, and intrahepatic lymphocytes were purified following mechanical disruption on a discontinuous Ficoll (Sigma Aldrich, St. Louis, United States) gradient.

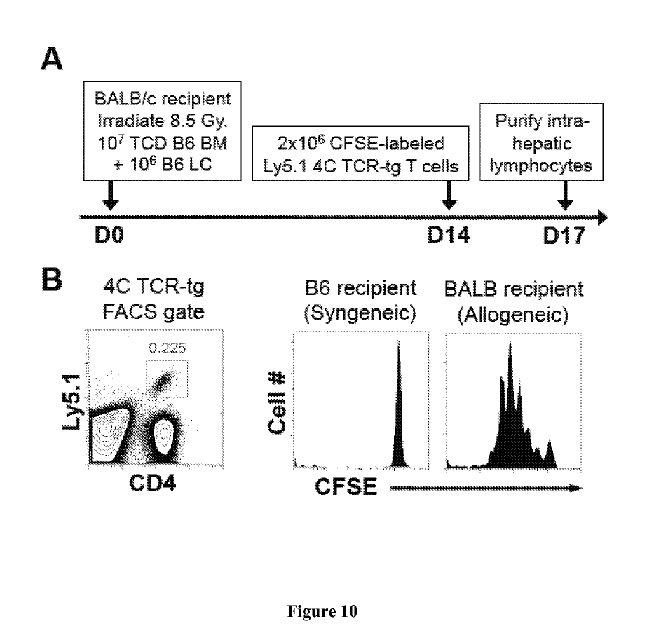

This model produces lethal GVHD within 30 days. At 14 days following transplant, 10⁶ CFSE-labeled T cell receptor transgenic (TCR-tg) T cells were adoptively transferred from the 4C TCR-tg mouse. The 4C mouse is in the B6 background and has TCR-tg CD4⁺ T cells with direct allospecificity against the BALB/c MHC class II molecule, I-A^{d22}. TCR-tg T cells were also transferred into recipients of syngeneic HSCT on D14 as a control for homeostatic proliferation in potentially lymphopenic hosts (FIG. 10A). As shown, in FIG. 10B, there was robust proliferation of the TCR-tg T cells, demonstrating the presence of recipient antigen presenting cells (APCs) during the period of peak serum HS levels.

### Example 7

### Serum HS Elevation is Associated with GVHD in Patients Undergoing Allo-HSCT

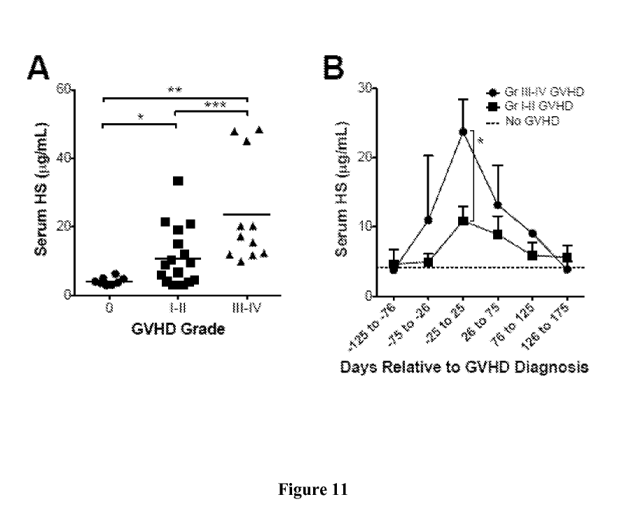

To investigate whether the elevation in serum HS levels observed in the mouse model of GVHD correlated to those found in the clinical setting, serum samples were obtained from human Allo-HSCT recipients with and without clinical and/or pathological evidence of acute GVHD (within 100 days of HSCT) as measured on a scale of grade 0 (no evidence of GVHD), grade I-II (mild GVHD), or grade III-IV (severe GVHD). Patient demographics, indication for HSCT, donor MHC match, conditioning regimens, maintenance immunosuppression and post-transplant infections were compared and found to be similar between groups (Table 1). Comparison of HS levels near the time of GVHD diagnosis demonstrated serum HS elevations that correlated with the severity of GVHD (grade 0, [HS]=4.22±0.39 μg/mL, n=8; grade I-II, [HS]=10.89±2.07 μg/mL, n=17; grade III-IV, [HS]=23.74±4.66 μg/mL, n=11) (FIG. 11A). Comparison of serum HS relative to the time of GVHD revealed a peak in serum HS levels that temporally correlated with the time of GVHD diagnosis (FIG. 11B).

TABLE 1
Characteristics of transplants according to degree of GVHD

| | GVHD | | | |
|---|---|---|---|---|
| | 0 | I-II | III-IV | p- |
| | (n = 8) | (n = 17) | (n = 11) | Value* |
| Recipient Factors | | | | |
| Age (yrs) | 48.0 ± 12.2 | 45.8 ± 13.3 | 44.5 ± 16.2 | 0.86 |
| Male | 87.5% | 58.8% | 72.7% | 0.38 |
| Caucasian race | 100% | 76.5% | 72.7% | 0.31 |
| HSCT Indication | | | | |
| Acute myeloid leukemia | 4 | 5 | 2 | 0.83^{#} |
| Acute lymphoblastic | 0 | 3 | 2 | |
| leukemia | | | | |
| Chronic lymphocytic | 1 | 0 | 3 | |
| leukemia | | | | |
| Hodgkin's lymphoma | 0 | 3 | 0 | |
| Myelodysplastic syndrome | 1 | 0 | 2 | |
| Aplastic Anemia | 1 | 2 | 0 | |
| Other | 1 | 4 | 2 | |
| Conditioning Regimen | | | | |
| Ablative | 3 | 4 | 4 | 0.79 |
| Non-ablative/reduced | 5 | 13 | 7 | |
| intensity | | | | |
| Total body irradiation | 1 | 1 | 2 | |
| Maintenance | | | | |
| Immunosuppression | | | | |
| Cellcept | 3 | 14 | 9 | 0.32 |
| Calcineurin inhibitor | 4 | 3 | 4 | |
| Prednisone | 1 | 3 | 2 | |
| Sirolimus | 0 | 0 | 1 | |
| None | 1 | 0 | 0 | |
| Donor-Recipient | | | | |
| MHC Match | | | | |
| 6/6 | 7 | 10 | 5 | 0.19‡ |
| 2-5/6 | 1 | 6 | 6 | |
| 0/6 | 0 | 1 | 0 | |
| Infection within 100 | | | | |
| days of transplant | | | | |
| CMV viremia | 2 | 10 | 7 | 0.50 |
| Bacteremia | 0 | 0 | 1 | |

*Kruskal-Wallis for continuous variables; Fisher's exact test for categorical variables.
^{#}Acute leukemia vs. others.
‡6/6 vs. 0-5/6 MHC match.

### Example 8

### HS is an Endogenous Stimulator of Alloimmunity and an Early Biomarker of Immune Injury in a Mouse Heart Transplant Model

Allogenic heart transplants were performed between C57BL/6 donors and (BALB/c x DBA.1)F1 recipients. Serum HS was measured by ELISA Immunohistologic staining for HS and CD3 was performed on heart tx tissue. Bone marrow derived dendritic cells (DCs) and purified T cells from WT-, MyD88- or TLR4-deficient mice were used in MLR assays to test for proliferation and cytokine production in response to HS. CD40 and CD80 expression on stimulated DCs was measured by FACS. T cell proliferation was determined by ³H-thymidine incorporation and CFSE dye dilution assay. HEK293 NF-kB-luciferase reporter cell lines stably expressing CD14, MD2, and TLR4 or TLR2 were used in assays of HS activity.

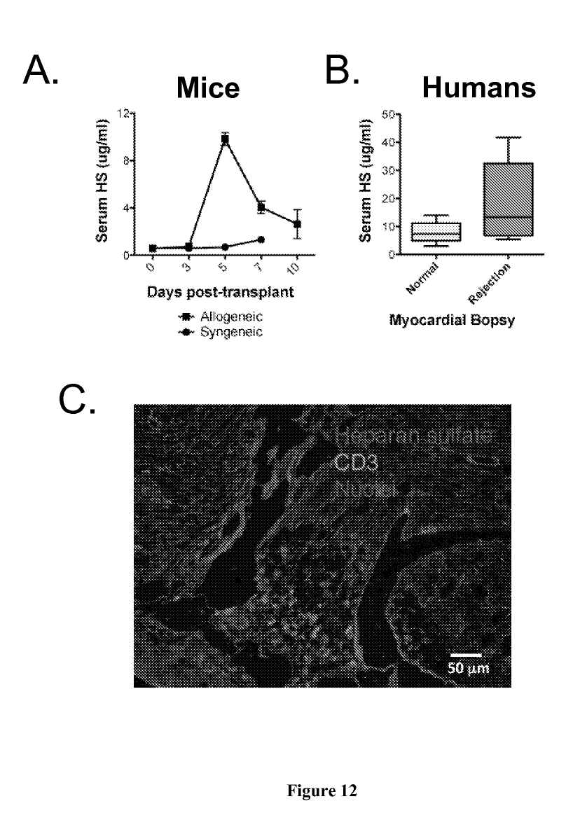

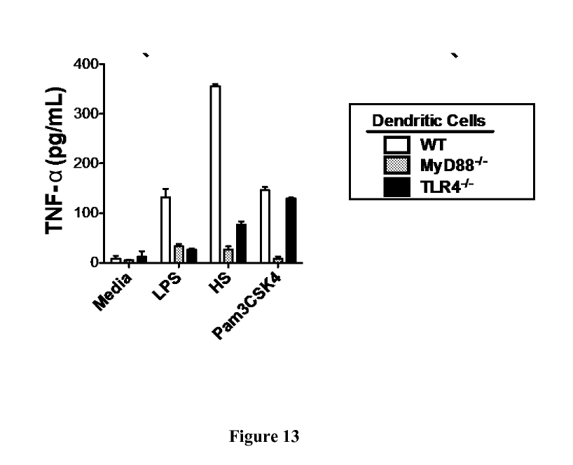

HS was increased in the serum of mice undergoing acute cardiac allograft rejection, but not in syngeneic controls (9.80.5 vs. 0.70.1 μg/ml, p=0.003). (FIG. 12A). These results were observed in human as well. (FIG. 12B). Tissue HS was decreased or absent in areas of focal T cell infiltration. (FIG. 12C). HS was found to up-regulate DC expression of CD40, CD80, IL-6, IL-12, and TNF-α in a TLR4- and MyD88-dependent manner. (FIG. 13). In MLR assays, HS increased allogeneic LC proliferation (CD4 & CD8) and IFNg production. The stimulation of LC was dependent on APC, but not T cell, expression of MyD88. HS stimulation depended on PI3K activity and caused NF-kB activation. HS stimulation of IL-8 production by CD14/MD2/TLR4 expressing HEK293 cells was specifically inhibited by heparanase, but not by the LPS inhibitor, polymyxin B.

These results demonstrate that HS is an innate immune stimuli of APCs that promotes alloimmunity. Additionally, blocking extracellular matrix breakdown may inhibit lymphocytic tissue infiltration and reduce T cell activation.

The results herein demonstrate that HS, an endogenous TLR4 ligand, was released during the onset of GVHD and may promote GVHD, which usually occurs in the absence of obvious exogenous TLR stimuli. The observations that inhibition of HS release by A1AT resulted in a significant improvement in GVHD and survival in mice, and that serum HS levels were directly correlated to the severity of GVHD in humans, demonstrate that blockade of HS release following Allo-HSCT provides an effective and novel strategy for the control of clinical GVHD.

In the setting of alloreactive T cell responses, there was no significant difference in proliferation of MyD88^{-/-} T cells stimulated with HS, compared to the WT T cell controls.

Instead, the HS-dependent enhancement of alloreactivity in vitro was mediated by activating the TLR4-MyD88 and TRIF pathway in APCs.

Recipient APC of hematopoietic lineage are rapidly depleted following Allo-HSCT. These studies determined if any recipient APC was present at the time of HS elevation in GVHD. Using an adoptive transfer model in which alloreactive donor-strain TCR-tg T cells with direct alloreactivity against recipient MHC class II are transferred to Allo-HSCT recipients, donor APCs are present when previous studies have shown near complete depletion of recipient hematopoietic APCs. This experiment provides evidence that recipient MHC class II expressing cells are present at the time of HS elevation.

Alternatively, HS may activate donor APCs that are capable of presenting recipient alloantigen to donor T cells through indirect antigen presentation. It has been suggested that myeloablative conditioning regimens such as chemotherapy and irradiation can cause injury to the bowel, which can release DAMPs and allow PAMP-producing bacterial to translocation across the bowel epithelium. However, clinical GVHD often occurs weeks or months following transplantation and the contribution of tissue damage from conditioning regimens is not clearly linkable to these episodes. In this study, the results demonstrate that HS did not become elevated in the serum as a consequence of irradiation, bone-marrow transplantation or from the reconstitution of syngeneic lymphocyte populations. Instead, it became highly elevated at the onset of clinical GVHD in the serum of recipients that received allogeneic lymphocytes. These observations indicate that HS release is related to the alloreactive T cell response involved in GVHD.

In conclusion, the results demonstrate that HS promotes alloreactive T cell responses in vitro. In mice, serum HS levels are acutely elevated at the onset of clinical GVHD following Allo-HSCT. Treatment with A lAT decreases HS levels, leading to a reduction in alloreactive T cell responses and an improvement in GVHD. Conversely, a HS mimetic that increases serum HS levels accelerates GVHD. In patients undergoing Allo-HSCT for hematologic malignancies, serum HS level elevations correlate with the severity of GVHD. These results identify a new role for HS in promoting acute GVHD following Allo-HSCT, and controlling clinical GVHD through modulation of HS release.

Any patents or publications mentioned in this specification are indicative of the levels of those skilled in the art to which the invention pertains. These patents and publications are herein incorporated by reference to the same extent as if each individual publication was specifically and individually indicated to be incorporated by reference.

One skilled in the art will readily appreciate that the present invention is well adapted to carry out the objects and obtain the ends and advantages mentioned, as well as those inherent therein. The present examples along with the methods described herein are presently representative of preferred embodiments, are exemplary, and are not intended as limitations on the scope of the invention. Changes therein and other uses will occur to those skilled in the art which are encompassed within the spirit of the invention as defined by the scope of the claims.

We claim:

1.-29. (canceled)

30. A method of treating or ameliorating an injurious condition associated with elevated heparan sulfate comprising administering an inhibitor that decreases serum heparan sulfate to a therapeutically effective level, to a subject that was the recipient of a transplanted organ, tissue, or cells.

31. A method of preventing an injurious condition associated with elevated heparan sulfate comprising administering an inhibitor that decreases serum heparan sulfate to a therapeutically effective level, to a subject that was the recipient of a transplanted organ, tissue, or cells.

32. The method of claim 30, where the inhibitor is heparanase.

33. The method of claims 30, where the inhibitor is a serine protease inhibitor.

34. The method of claim 33, where the serine protease inhibitor is al-antitrypsin.

35. The method of claim 30, where the transplant is a solid organ selected from the group consisting of heart, lung, kidney, liver, pancreas, thymus, and intestine.

36. The method of claim 35, where the solid organ transplant is a heart.

37. The method of claim 35, where the solid organ transplant is a kidney.

38. The method of claim 30, where the transplant is tissue, selected from the group consisting of bone, tendon, cornea, skin, heart valve, and veins.

39. The method of claim 30, where the transplant are cells, selecting from the group consisting of hematopoietic stem cells derived from bone marrow, peripheral blood, and umbilical cord blood.

40. The method of claim 39, where the transplanted cells are allogeneic hematopoietic stem cells.

41. The method of claim 30, where the injurious condition is an innate immune injury.

42. The method of claim 41, where the innate immune injury comprises inflammation, graft-versus-host-disease, or acute allograft rejection.

43. The method of claim 30, where an immunosuppressant selected from the group consisting of cellcept, calcineurin inhibitor, prednisone, and sirolimus is administered in combination with the inhibitor.

44. The method of claim 30, where a conditioning regimen selected from the group consisting of ablative, non-ablative/reduced intensity, and total body irradiation is administered in combination with the inhibitor.

45. The method of claim 30, where the therapeutically effective level of serum heparan sulfate is in a concentration of about 2μg/mL to about 15 μg/mL.

46. A method of diagnosing an injurious condition that is associated with elevated heparan sulfate in a subject that was the recipient of a transplanted organ, tissue, or cells, by collecting a biological sample from the subject and determining the serum concentration of heparan sulfate, where the concentration of heparan sulfate directly correlates with the severity of the heparan sulfate-mediated immune injury.

47. The method of claim 46, where the heparan sulfate-mediated immune injury is graft-versus-host disease, and where the severity of the graft-versus-host disease is determined by a serum heparan sulfate concentration of about 2 μg/mL to about 30 μg/mL.

48. The method of claim 46, where the heparan sulfate-mediated immune injury is acute cardiac allograft rejection, and where the severity of the acute cardiac allograft rejection is determined by a serum heparan sulfate concentration of about 10 μg/mL to about 40 μg/mL.

49. The method of claim 46, where the biological sample is collected from the subject 7-100 days following cell transplant comprising allogeneic hematopoietic stem cell transplantation.

50. A composition comprising an inhibitor that decreases serum heparan sulfate to a therapeutically effective level in a transplant recipient subject, and a pharmaceutically acceptable carrier.

51. The composition according to claim 50, wherein the inhibitor is a serine protease inhibitor.

52. The composition according to claim 51, wherein the serine protease inhibitor is α1-antitrypsin.

53. The composition according to claim 50, wherein the inhibitor is heparanase.
